(19) United States
(12) Patent Application Publication
(10) Pub. No.: US 2017/0087069 A1
(43) Pub. Date: Mar. 30, 2017

(54) TOPICAL COMPOSITIONS

(71) Applicant: DSM IP ASSETS B.V., Heerlen (NL)

(72) Inventors: Aline HUEBER, Kaiseraugst (CH); Anne JANSSEN, Kaiseraugst (CH); Christine SAECKER, Kaiseraugst (CH)

(21) Appl. No.: 15/126,385

(22) PCT Filed: Mar. 24, 2015
(86) PCT No.: PCT/EP2015/056262

§ 371 Date: Sep. 15, 2016

(30) Foreign Application Priority Data
Mar. 31, 2014 (EP) 14162823.0

(51) Int. Cl. A61K 8/34; A61K 8/92; A61K 8/25; A61Q 17/04; A61K 8/49; A61K 8/06; A61K 8/55; A61K 8/73; A61K 8/29

(52) U.S. Cl. CPC A61K 8/345; A61K 8/731; A61K 8/92; A61K 8/25; A61K 8/29; A61K 8/4946; A61K 8/062; A61K 8/55; A61Q 17/04; A61K 2800/20

(57) ABSTRACT

The present invention relates to a topical composition comprising glycerin, microcrystalline cellulose and/or microcrystalline wax and silica beads having a particle size Dᵥ0 of greater 0.3 μm, a Dᵥ100 of less than 35 μm, a Dᵥ50 selected in the range of 9 to 15 μm and an oil absorption capacity selected in the range of 1.2-2.5 cc/g. Furthermore the invention relates to the use of such beads in combination with microcrystalline cellulose and/or microcrystalline wax to ameliorate the short and long-term sensory properties of a topical composition comprising glycerin.

The present invention relates to a topical composition comprising glycerin, microcrystalline cellulose and/or microcrystalline wax and silica beads having a particle size Dᵥ0 of greater 0.3 μm, a Dᵥ100 of less than 35 μm, a Dᵥ50 selected in the range of 9 to 15 μm and an oil absorption capacity selected in the range of 1.2-2.5 cc/g. Furthermore the invention relates to the use of such beads in combination with microcrystalline cellulose and/or microcrystalline wax to ameliorate the short and long-term sensory properties of a topical composition comprising glycerin.

Glycerin is a humectant that is commonly added to cosmetic compositions such as sun care products. However, incorporated into topical compositions, glycerin can impart a very sticky feeling in particular when used in higher amounts. This drawback is even more pronounced in sun-care products (sunscreens) which due to the presence of oil soluble UV-filters already tend to be sticky.

Thus, there is an ongoing need for substances which reduce the stickiness of glycerin in topical compositions such as in particular in sun care products comprising oil soluble UV-filter(s) and thus contribute to an overall improved skin feel.

Surprisingly it has been found that the addition of specific silica beads in combination with microcrystalline cellulose and/or microcrystalline wax to a topical composition comprising glycerin significantly reduced the stickiness of such compositions. Furthermore, the topical compositions exhibited a significantly enhanced absorbency and a superior dry touch.

Thus, the present invention relates to a topical composition comprising glycerin, microcrystalline cellulose and/or microcrystalline wax and silica beads having a particle size Dᵥ0 of greater 0.3 μm, a Dᵥ100 of less than 35 μm, a Dᵥ50 selected in the range of 9 to 15 μm and an oil absorption capacity selected in the range of 1.2-2.5 cc/g.

Another subject matter of the invention is directed to a method reducing the stickiness of a topical composition comprising glycerin, said method comprising the step of adding to the topical composition silica beads having a particle size Dᵥ0 of greater than 0.3 μm, a Dᵥ100 of less than 35 μm, a Dv50 selected in the range of 9 to 15 μm and an oil absorption capacity selected in the range of 1.2-2.5 cc/g in combination with microcrystalline cellulose and/or microcrystalline wax and appreciating the effect.

In a further embodiment the invention relates to the use of silica beads having a particle size Dᵥ0 of greater than 0.3 μm, a Dᵥ100 of less than 35 μm, a Dᵥ50 selected in the range of 9 to 15 μm and an oil absorption capacity selected in the range of 1.2-2.5 cc/g in combination with microcrystalline cellulose and/or microcrystalline wax to reduce the stickiness of a topical composition comprising glycerin.

In an additional embodiment the invention relates to the use of silica beads having a particle size Dᵥ0 of greater than 0.3 μm, a Dᵥ100 of less than 35 μm, a Dᵥ50 selected in the range of 9 to 15 and an oil absorption capacity selected in the range of 1.2-2.5 cc/g in combination with microcrystalline cellulose and/or microcrystalline wax to enhance dry touch feel of a topical composition comprising glycerin.

Another subject matter of the invention is directed to a method reducing the stickiness and/or to enhance the absorbency of a topical composition comprising glycerin and silica beads having a particle size Dᵥ0 of greater than 0.3 μm, a Dᵥ100 of less than 35 μm, a Dv50 selected in the range of 9 to 15 μm and an oil absorption capacity selected in the range of 1.2-2.5 cc/g said method comprising the step of adding to the topical composition microcrystalline cellulose and/or microcrystalline wax and appreciating the effect.

In another embodiment the invention relates to the use of microcrystalline cellulose and/or microcrystalline wax to reduce the stickiness and/or to enhance the absorbency of a topical composition comprising glycerin and silica beads having a particle size Dᵥ0 of greater than 0.3 μm, a Dᵥ100 of less than 35 μm, and a Dᵥ50 selected in the range of 9 to 15 μm and an oil absorption capacity selected in the range of 1.2-2.5 cc/g.

In all embodiments of the present invention, the amount of the silica beads is preferably selected in the range of 0.5 to 10 wt.-%, more preferably in the range of 1 to 5 wt.-%, and most preferably in the range of 2 to 4 wt.-% based on the total weight of the composition.

In all embodiments of the present invention, the total amount of the microcrystalline cellulose and/or microcrystalline wax is preferably selected in the range of 0.1 to 10 wt.-%, more preferably in the range of 0.5 to 5 wt.-%, and most preferably in the range of 1 to 3 wt.-% based on the total weight of the composition. It is furthermore preferred that either microcrystalline cellulose alone (i.e. in the absence of microcrystalline wax) or microcrystalline cellulose in combination with microcrystalline wax is used in the topical compositions according to the present invention. Most preferably in all embodiments of the present invention microcrystalline cellulose is used in the absence of microcrystalline wax in the topical compositions as this results in the best performance in view of reduced stickiness and improved absorbency.

In all embodiments of the present invention, the amount of glycerin is preferably selected in the range of 1 to 20 wt.-%, more preferably in the range of 3 to 15 wt.-%, and most preferably in the range of 5 to 10 wt.-% based on the total weight of the composition.

The porous silica beads used according to the present invention may be prepared from sodium silicate by emulsion polymerization according to standard procedures such as e.g. via the sol-gel method. They silica beads may be used as such or may be further coated with a suitable coating agent such as e.g. a silicone oil. Suitable silicone oils include in particular non-volatile silicone oils such as dialkyl and alkyl aryl siloxanes as well as alkoxylated and/or aminated derivatives thereof, dihydroxypolydimethylsiloxanes and polyphenylalkylsiloxanes. Preferred silicone oils encompass dimethicone, dimethiconol, dimethicone copolyol, phenyl trimethicone, methicone, simethicone as well as mixtures thereof. Most preferably the silica beads are either uncoated or coated with dimethicone. In a preferred embodiment the silica beads used according to the present invention are uncoated.

The particle size of the beads according to the invention (in volume %) is determined by a Coulter LS13320 or Malvern Mastersizer 2000 according to standard methods in the art. In number % the average particle size Dₙ50 ranges from 9 to 15 μm.

In all embodiments of the present invention the oil absorption capacity of the silica beads is preferably selected in the range of 1.2 to 2.0 cc/g and most preferably in the range of 1.3 to 1.8 cc/g.

The oil absorption capacity refers to the weight of a specific oil absorbed by a material, determined by a specified method. It includes the oil absorption capacity of the dry particles existing between the inherent voids within and on the surface of the particles. The oil absorption capacity as referred to in the present invention is determined at 23° C. by weighting 2 g of the respective silica beads into a 20 ml beaker glass. Then liquid paraffin (Paraffinum Perliquidum PH.EUR. CAS 8042-47-5) is added. After addition of 4 to 5 drops of paraffin to the powder, mixing is performed using a spatula, and addition of paraffin is continued until conglomerates of oil and powder have formed. From this point, the paraffin is added one drop at a time and the mixture is then triturated with the spatula. The addition of oil is stopped when the loose and dry powder completely disappears and a highly viscous white to transparent homogeneous gel is obtained. The oil absorption capacity (cc/g) is then calculated by the volume of paraffin used (in cc) per g of silica beads.

Particularly suitable porous silica beads according to the present invention are e.g. obtainable as VALVANCE™ Touch 210 at DSM Nutritional Products Ltd Kaiseraugst.

Particularly suitable microcrystalline cellulose according to the present invention is the isolated, crystalline portion of cellulose fibers from wood pulp which can be used in colloidal (i.e. co-processed with a soluble hydrocolloid) or non-colloidal form. Particularly advantageous microcrystalline cellulose according to the present invention is available as Avicel range at FMC Biopolymer US. In a particular preferred embodiment, the microcrystalline cellulose is used in colloidal form having a particle size of <1 um, a rod-like particle shape. The initial viscosity is preferably selected in the range of 30 to 150 cps as a 1 to 3% dispersion. Even more preferably, the microcrystalline cellulose has additionally a sieve fraction of +60 mesh of not more than 0.1% wt and +325 mesh not more than 75% wt. Most preferably, the microcrystalline cellulose is Avicel 611 (INCI Microcrystalline Cellulose and Cellulose gum, having a particle size of <1 um, a rod-like particle shape and a sieve fraction+60 mesh of not more than 0.1% wt and +325 mesh not more than 75% wt. and an initial viscosity of 50 to 151 cps (2.6% dispersion))

Suitable microcrystalline waxes according to the present invention are of mineral origin and may be produced by de-oiling petrolatum, as part of the petroleum refining process. Microcrystalline waxes generally contain a high percentage of isoparaffinig (branched) hydrocarbons and naphtenic hydrocarbons. They are characterized by the fineness of its crystals in contrast to the larger crystal of paraffin wax. They consist of high molecular weight saturated aliphatic hydrocarbons. Particularly suitable microcrystalline wax according to the present invention is Paracera™ M available at Paramel or Kahlwax 1847 available at Kahl GmbH & Co.

In another embodiment of the present invention the topical compositions are light protective preparations (sunscreens) comprising additionally at least one oil soluble UV-filter substance.

The total amount of the at least one additional oil soluble UV-filter substance is preferably selected in the range of about 0.1 to 25 wt.-%, more preferably in the range of about 1 to 20 wt.-%, most preferably in the range of 5 to 15 wt.-% based on the total weight of the topical composition.

Particularly preferred oil soluble UV-filter substances encompass dibenzoylmethane derivatives such as butyl methoxydibenzoylmethane (e.g. commercially available as PARSOL® 1789 at DSM Nutritional Products Ltd), organopolysiloxanes functionalized with at least one UV-light absorbing group such as polysilicone-15 (e.g. commercially available as PARSOL® SLX at DSM Nutritional Products Ltd), diphenylacrylates such as octocrylene (e.g. commercially available as PARSOL® 340 at DSM Nutritional Products Ltd) and cinnamates such as octyl methoxycinnamate (e.g. commercially available as PARSOL® MCX at DSM Nutritional Products Ltd) as well as mixtures thereof.

In a preferred embodiment, the topical compositions according to the present invention comprise as oil soluble UV-filter substances only butyl methoxydibenzoylmethane, octocrylene and polysilicone-15.

In the light protective preparations according to the present invention the amount butyl methoxydibenzoylmethane is preferable selected in the range of about 2 to 8 wt.-%, in particular in the range of about 2.5 to 6 wt.-%, most particular in the range of about 3 to 5 wt.-%, the amount octocrylene is preferable selected in the range of about 2 to 15 wt.-%, in particular in the range of about 4 to 10 wt.-%, most particular in the range of about 6 to 9 wt.-% and the amount of polysilicone-15 is preferable selected in the range of about 0.1 to 5 wt.-%, in particular in the range of about 0.5 to 4 wt.-%, most particular in the range of about 1 to 3 wt.-%, based on the total weight of the topical composition.

In a further advantageous embodiment the light protective preparations comprise next to the at least one oil soluble UV-filter substance further at least one water soluble and/or inorganic UV-filter substance.

Preferably the water soluble UV-filter substance is selected from the group consisting of 2-phenylbenzimidazol-sulphonic acid or 2,2′-(1,4-phenylene)bis-1H-benzimidazole-5,7-disulfonic acid as well as the respective salts thereof. Preferably the water soluble UV-filter substance is 2-phenylbenzimidazol-sulphonic acid (e.g. commercially available as PARSOL® HS at DSM Nutritional Products Ltd).

The total amount of the water soluble UV-filter substance(s) in the topical composition according to the invention is preferably selected in the range of about 0.1 to 5 wt.-%, more preferably in the range of about 0.5 to 4 wt.-%, most preferably in the range of 1 to 3 wt.-% based on the total weight of the topical composition.

Inorganic UV-filter substances encompass UV-light absorbing pigments such as e.g. microparticulated Zink oxide or Titanium dioxide. The term “microparticulated” refers to a particle size from about 5 nm to about 200 nm, particularly from about 15 nm to about 100 nm. The particles may also be coated by other metal oxides such as e.g. aluminum or zirconium oxides or by organic coatings such as e.g. polyols, methicone, aluminum stearate, alkyl silane. Such coatings are well known in the art. In all embodiment of the present invention, preferably the inorganic UV-filter substance is titanium dioxide having an inner silica and an outer dimethicone coating which is commercially available as PARSOL® TX at DSM Nutritional Products Ltd) is used.

The total amount of the inorganic UV-filter substance(s) in the topical composition according to the invention is preferably selected in the range of about 0.1 to 10 wt.-%, more preferably in the range of about 0.5 to 8 wt.-%, most preferably in the range of 2 to 5 wt.-% based on the total weight of the topical composition.

In particular preferred embodiment the invention relates to topical compositions comprising glycerin, microcrystalline cellulose and/or microcrystalline wax, silica beads having a particle size Dᵥ0 of greater 0.3 μm, a Dᵥ100 of less than 35 μm, a Dv50 selected in the range of 9 to 15 μm and an oil absorption capacity selected in the range of 1.2-2.5 cc/g, octocrylene, butyl methoxydibenzoylmethane, polysilicone-15, 2-phenylbenzimidazol-sulphonic acid and titanium dioxide coated having an inner silica and an outer dimethicone coating. In these composition it is further preferred that the amount of octocrylene is selected in the range of 5 to 10 wt.-%, the amount of butyl methoxydibenzoylmethane is selected in the range of 3-5 wt.-%, the amount of polysilicone-15 is selected in the range of 1-3 wt.-% and the amount of coated titanium dioxide is selected in the range of 2-4 wt.-%, based on the total weight of the composition.

The term “topical” as used herein is understood to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, and keratinous fibers. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations.

Examples of skin care preparations are, in particular, light protective preparations (sunscreens), anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), as well as skin lightening preparations as well as BB and CC Creams.

In a particular embodiment the topical compositions according to the invention are skin care preparations, such as (body) milks, lotions, hydrodispersions, foundations, creams, creamgels, serums, toners or gels.

The topical compositions according to the present invention may be in the form of a suspension or dispersion in solvents or fatty substances, or alternatively in the form of an emulsion or micro emulsion (in particular of oil-in-water (O/W) or water-in-oil (W/O) type, silicone-in-water (Si/W) or water-in-silicone (W/Si) type, PIT-emulsion, multiple emulsion (e.g. oil-in-water-in oil (O/W/O) or water-in-oil-in-water (W/O/W) type), pickering emulsion, hydrogel, alcoholic gel, lipogel, one- or multiphase solution or vesicular dispersion or other usual forms, which can also be applied by pens, as masks or as sprays.

In one embodiment, the topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art.

If the topical composition according to the invention is an O/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, Glyceryl Stearate Citrate, Glyceryl Stearate SE (self-emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Cetearyl Glucoside, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one O/W, respectively Si/W emulsifier is preferably used in an amount of 0.5 to 10 wt.-%, in particular in the range of 0.5 to 6 wt.-% such as more in particular in the range of 0.5 to 5 wt.-% such as most in particular in the range of 1 to 4 wt.-%, based on the total weight of the composition.

Particular suitable O/W emulsifiers according to the present invention encompass phosphate esters emulsifier of formula (II)

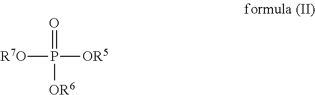

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, 06-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate.

A particular suitable O/W emulsifier to be used in the topical compositions according to the invention is potassium cetyl phosphate e.g. commercially available as Amphisol® K at DSM Nutritional Products Ltd Kaiseraugst.

Further suitable O/W emulsifiers are polyethyleneglycol (PEG) esters or diesters such as e.g. [INCI Names] PEG-100 Stearate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate.

Particularly preferred according to the present invention is PEG-100 Stearate sold under the tradename Arlacel™ 165 (INCI Glyceryl Stearate (and) PEG-100 Stearate) by Croda.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

In particular embodiment, the invention relates to topical compositions with all the definitions and preferences given herein in the form of O/W emulsions comprising an oily phase dispersed in an aqueous phase in the presence of an O/W emulsifier wherein the O/W emulsifier is potassium cetyl phosphate.

The compositions in form of O/W emulsions according to the invention can be provided, for example, in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the topical compositions constitute cosmetic composition and are intended for topical application to the skin.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into such compositions. Such cosmetic ingredients commonly used in the skin care industry, which are suitable for use in the compositions of the present invention, are e.g. described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of the cosmetic and dermatological adjuvants and additives can based on the desired product easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7.5. The pH can easily be adjusted as desired with suitable acids such as e.g. citric acid or bases such as sodium hydroxide (e.g. as aqueous solution), Triethanolamine (TEA Care), Tromethamine (Trizma Base) and Aminomethyl Propanol (AMP-Ultra PC 2000) according to standard methods in the art.

The amount of the topical composition to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

The following examples are provided to further illustrate the compositions and effects of the present invention. These examples are illustrative only and are not intended to limit the scope of the invention in any way.

### EXAMPLE 1

### Oil Absorption Capacity VALVANCE™TOUCH 210

About 2.0 g-2.2 g of the silica beads was weighted in a small beaker glass (20 ml) at 23° C. After addition of 4 to 5 drops of paraffin to the powder, mixing was performed using a spatula, and addition of paraffin is continued until conglomerates of oil and powder have formed. From this point, the paraffin was added one drop at a time and the mixture was then triturated with the spatula. The addition of oil was stopped when the loose and dry powder completely disappeared and a highly viscous white to transparent homogeneous gel was obtained. The oil absorption capacity (cc/g) was then calculated by the volume of paraffin used (in cc) per g of silica beads to be 1.58 cc/g.

### EXAMPLE 2

### Sensory Evaluation I

### 2.1 Formulations

TABLE 1
Sunscreen SPF 30

| Ingredients | INCI | % w/w |
|---|---|---|
| PARSOL ® 1789 | Butyl Methoxydibenzoylmethane | 4.00 |
| PARSOL ® 340 | Octocrylene | 8.00 |
| PARSOL ® SLX | Polysilicone-15 | 1.50 |
| PARSOL ® TX | Titanium Dioxide & Silica & Dimethicone | 3.00 |
| AMPHISOL ® K | Potassium Cetyl Phosphate | 2.00 |
| | Cetearyl Alcohol | 0.80 |
| | C12-15 Alkyl Benzoate | 3.00 |
| | Butylene Glycol Dicaprylate/Dicaprate | 4.00 |
| | Dicaprylyl Carbonate | 3.00 |
| | Phenoxyethanol & Ethylhexyl Glycerin | 1.00 |
| | Glycerin | 5.00 |
| | Xanthan Gum | 0.30 |
| | Microcrystalline Cellulose | 1.20 |
| | Disodium EDTA | 0.10 |
| | Aqua | ad 100 |
| | Tromethamine | 0.25 |
| PARSOL ® HS | Phenylbenzimidazole Sulfonic Acid | 1.50 |
| | Tromethamine | 0.75 |
| | Aqua | 10.00 |
| VALVANCE ™ | Silica | 1.50 |
| Touch 210 | | |

TABLE 2
Sunscreen: SPF 50⁺

| Ingredients | INCI | % w/w |
|---|---|---|
| PARSOL ® 1789 | Butyl Methoxydibenzoylmethane | 5.00 |
| PARSOL ® 340 | Octocrylene | 14.00 |
| PARSOL ® SLX | Polysilicone-15 | 2.00 |
| PARSOL ® TX | Titanium Dioxide & Silica & Dimethicone | 3.00 |
| AMPHISOL ® K | Potassium Cetyl Phosphate | 2.00 |
| | Cetearyl Alcohol | 1.20 |
| | C12-15 Alkyl Benzoate | 4.00 |
| | Diisopropyl Sebacate | 8.00 |
| | Phenoxyethanol & Ethylhexyl Glycerin | 1.00 |
| | Glycerin | 5.00 |
| | Xanthan Gum | 0.20 |
| | Microcrystalline Cellulose | 0.60 |
| | Disodium EDTA | 0.10 |
| | Aqua | ad 100 |
| | Tromethamine | 0.25 |
| PARSOL ® HS | Phenylbenzimidazole Sulfonic Acid | 2.00 |
| | Tromethamine | 1.00 |
| | Aqua | 10.00 |
| VALVANCE ™ | Silica | 2.00 |
| Touch 210 | | |

### 2.2 Sensory Evaluation

The formulations as outlined in Table 1 and 2 above were tested in a blind study with a trained sensorial panel consisting of 8 persons under the following conditions: The evaluation takes part on the inner forearm; the panel leader applies 50 μL of the respective sample.

Evaluator spreads the product within a defined circle of 5 cm diameter using index or middle finger, circular motion, rate of 2 rotations/second. This is the so called rub-out phase. After the rub-out phase the stickiness is assessed relative to a set of standards. The stickiness is scaled on a scale from 0 to 100 in comparison to trained standards. Data are median values. The stickiness values are assessed immediately after application as well as after 20 min. As can be retrieved from table 4, the compositions according to the present invention showed a significantly reduced stickiness directly after application as well as after 20 min compared to commercially available sunscreens (listed in table 3).

TABLE 3
Stickiness values for commercially available
sunscreens with SPF 30 and 50

| | | Stickiness | |
|---|---|---|---|
| | References | Immediate | After 20 min |
| 1. | Garnier Ambre Solaire UV Sensitive | 14 | 10 |
| | Sonnenschutzmilch SPF 50⁺ | | |
| 2. | Nivea sun Kids Pure & Sensitive | 17 | 10 |
| | Sonnenspray SPF 50⁺ | | |
| 3. | Soltan Once Kids Lotion 50⁺ | 17 | 8 |
| 4. | Ombia Suncare Spray 30 | 15 | 5 |

1. Garnier Ambre Solaire, UV Sensitive Sonnenschutzmilch SPF 50⁺ (Company: L'Oreal; purchased August 2012 in Germany); INCI: Aqua, Octocrylene, Propylene Glycol, Glycerin, Titanium dioxide, C12-15 Alkyl benzoate, Cyclopentasiloxane, Butyl Methoxydibenzoylmethane, Drometrizole Trisiloxane (Benzotriazol), Isohexadecan, Glyceryl Stearate, Potassium Cetyl Phosphate, VP/Eicosene Copolymer, Tocopherol, Phenoxyethanol, PEG-100 Stearate, Ethylparaben, Triethanolamine, Stearic Acid, Chlorphenesin, Dimethicone, Xanthan Gum, Propylparaben, Terephthalylidene Dicamphor Sulfonic Acid, Acrylates/c10-30 Alkyl Acrylate Crosspolymer, Rosa Canina Fruit Oil, Disodium Edta, Methylparaben., Butylparaben, Aluminum Hydroxide, Palmitic Acid, FIL B50529/1.
2. Nivea sun Kids Pure & Sensitive Sonnenspray SPF 50⁺ (Company: Beiersdorf, purchased July 2012 in Germany); INCI: Aqua, Glycerin, Octocrylene, Alcohol Denat., Butylene Glycol Dicaprylate/Dicaprate, Butyl Methoxydibenzoylmethane, Bis-Ethylhexyloxyphenol Methoxyphenyl Triazine, Homosalate, Titanium Dioxide, Ceteareth-20, C18-36 Acid Triglyceride, Methylpropanediol, Sodium Phenylbenzimidazole Sulfonate, Diethylhexyl Butamido Triazone, Ethylhexyl Methoxycinnamate, Tocopheryl Acetate, Ethylhexylglycerin, PVP/Hexadecene Copolymer, Acrylates/C10-30 Alkyl Acrylate Crosspolymer, Trimethoxycaprylylsilane, Trisodium EDTA, BHT
3. Soltan Once Kids 8 Hour Play Hypo-allergenic Suncare Lotion SPF 50+ (Company: Boots, purchased July 2012 in Switzerland); INCI: Aqua, C12-15 alkyl benzoate, Methylene bis-benzotriazolyl tetramethylbutylphenol (nano), Butyl methoxydibenzoyl-methane, Butylene glycol, Octocrylene, Tridecyl salicylate, PVP/eicosene copolymer, Bisethylhexyloxyphenol methoxyphenyl triazine, C18-36 acid glycol ester, Polyglyceryl-3 methylglucose distearate, Titanium dioxide (nano), Polysilicone-15, Decyl glucoside, Phenoxyethanol, Diethylhexyl butamido triazone, Potassium cetyl phosphate, Parfum, Tocopheryl acetate, Vitis vinifera seed oil, Acrylates/vinyl isodecanoate crosspolymer, Glycerin, Dimethyl oxazolidine, Propylene glycol, Potassium hydroxide, Xanthan gum, Tetrasodium EDTA, Manganese dioxide, Panax ginseng extract, Sodium ascorbyl phosphate, Aminomethyl propanol.
4. Ombia Suncare Spray 30 (Company: Aldi; purchased July 2012 in Germany); INCI: Aqua, Octocrylene, Alcohol, Dibutyl Adipate, Butyl Methoxydibenzoylemethane, Glyzerin, Titanium Dioxide, C12-15 Alkyl Benzoate, VP/Eicosene, Copolymer, Diethylhexyl, Methoxyphenyl, Triazine, Bisabolol, Acrylates/C10-30 Alkyl Acrylate Crosspolymer, Disodium Edta, Ethylhexyglyerin, Silica, Dimethicone.

TABLE 4
Stickiness values for topical compositions
according to the present invention

Stickiness

| Standard | Immediate | After 20 min |
|---|---|---|
| Sunscreen SPF 30 (Table 1) | 6 | 1 |
| Sunscreen SPF 50+ (Table 2) | 6 | 2 |

As can be retrieved from table 4, the topical compositions according to the present invention exhibit a significantly reduced stickiness directly after application as well as after 20 min compared to common market products.

### EXAMPLE 3

### Sensory Evaluation II

The formulations as outlined in Table 4 were tested in a blind study with a trained sensorial panel consisting of 8 persons under the following conditions:

The evaluation takes part on the inner forearm; the panel leader applies 50 μL of the respective sample.

Evaluator spreads the product within a defined circle of 5 cm diameter using index or middle finger, circular motion, rate of 2 rotations/second. This is the so called rub-out phase. During this rub-out phase the rotations are counted until the product starts to absorb into skin. The amount of rotations gives a clear number how long a cosmetic product needs to absorb. The absorbency is scaled on a scale from 0 to 100 in comparison to trained standards. The smaller the value the faster absorbs the product. After the rub-out phase the stickiness is assessed relative to a set of standards. The stickiness is scaled on a scale from 0 to 100 in comparison to trained standards. Data are median values. The stickiness values are assessed after 20 min. As can be retrieved from table 4, the addition of microcrystalline wax, respectively microcrystalline cellulose leads to a significantly reduction in the stickiness after 20 min compared to the base and to the reference comprising the silica beads. Furthermore, the absorbency is also significantly improved by the addition of microcrystalline wax, respectively microcrystalline cellulose. In addition, all samples according to the invention exhibit an improved dry touch compared to the base as well as to the reference.

TABLE 4

| | | INCI | Base | Ref | Inv 1 | Inv 2 |
|---|---|---|---|---|---|---|
| | Ingredients | Name | % w/w | | | |
| A | Water dem. | Aqua | Ad 100 | Ad 100 | Ad 100 | Ad 100 |
| | Glycerin | Glycerin | 11.60 | 11.60 | 11.60 | 11.60 |
| | 1,23 (86.5%) | | | | | |
| | Ph. Eur. | | | | | |
| | Avicel | Micro- | — | — | 2.50 | — |
| | PC 611 | crystalline | | | | |
| | | cellulose | | | | |
| | Kahlwax | Hydroge- | — | — | — | 2.50 |
| | 1847 | nated micro- | | | | |
| | | crystalline | | | | |
| | | wax | | | | |
| B | AMPHISOL ® | Potassium | 1.50 | 1.50 | 1.50 | 1.50 |
| | K | cetyl | | | | |
| | | phosphate | | | | |
| | Lanette O | Cetearyl | 3.45 | 3.45 | 3.45 | 3.45 |
| | | alcohol | | | | |
| | Myritol | Caprylic/ | 8.00 | 8.00 | 8.00 | 8.00 |
| | 318 | capric tri- | | | | |
| | | glyceride | | | | |
| | Isopropyl- | Isopropyl | 8.00 | 8.00 | 8.00 | 8.00 |
| | palmitate | palmitate | | | | |
| | PARSOL ® | Octocrylene | — | — | — | — |
| | 340 | | | | | |
| C | VALVANCE | Slica | — | 3.00 | 3.00 | 3.00 |
| | Touch 210 | | | | | |
| D | Phenonip | | 0.50 | 0.50 | 0.50 | 0.50 |
| | XB | | | | | |
| | Stickiness | | 1.78 | 0.84 | 0.48 | 0.63 |
| | after 20 min | | | | | |
| | % reduction | | | 52.8% | 73.0% | 64.6% |
| | in stickiness | | | | | |
| | vs. Base | | | | | |
| | % reduction | | | | 43% | 25% |
| | in stickiness | | | | | |
| | vs. Ref | | | | | |
| | Absorbency | | 90 | 93 | 70 | 74 |
| | % improve- | | | −3% | 22% | 18% |
| | ment in | | | | | |
| | absorbency | | | | | |
| | vs Base | | | | | |
| | % improve- | | | | 25% | 20% |
| | ment in | | | | | |
| | absorbency | | | | | |
| | vs Ref | | | | | |

### EXAMPLE 4

### Sunscreen: SPF 50

| | Tradename | INCI | Wt.-% |
|---|---|---|---|
| A | PARSOL 1789 | Butyl Methoxydibenzoylmethane | 3.00 |
| | PARSOL 340 | Octocrylene | 2.70 |
| | PARSOL HMS | Homosalate | 15.00 |
| | PARSOL EHS | Ethylhexyl Salicylate | 5.00 |
| | PARSOL SLX | Polysilicone-15 | 0.90 |
| | PARSOL MCX | Ethylhexyl Methoxycinnmate | 3.00 |
| | Eusolex 4360 | Benzophenone-3 | 5.00 |
| | Amphisol K | Potassium Cetyl Phosphate | 2.00 |
| | Lanette O | Cetearyl Alcohol | 1.20 |
| | Paracera M | Microcrystalline Wax | 2.00 |
| | SilCare Silicone | Caprylyl Methicone | 2.00 |
| | 41M15 | | |
| | BHT | BHT | 0.05 |
| | Euxyl PE 9010 | Phenoxyethanol & Ethylhexyl | 1.00 |
| | | Glycerin | |
| B | Glycerin 86.5% | Glycerin | 5.00 |
| | Keltrol CG SFT | Xanthan Gum | 0.20 |
| | Avicel PC 611 | Microcrystalline Cellulose | 0.60 |
| | Edeta BD | Disodium EDTA | 0.10 |
| | Water demin. | Aqua | Ad 100 |
| D | Allianz OPT | Acrylates (and) C12-22 | 2.00 |
| | | Alkylmethacrylate Copolymer | |
| | VALVANCE ™ | Silica | 2.00 |
| | Touch 210 | | |

1. A topical composition comprising glycerin, microcrystalline cellulose and/or microcrystalline wax and silica beads having a particle size Dᵥ0 of greater 0.3 μm, a Dᵥ100 of less than 35 μm, a Dv50 selected in the range of 9 to 15 μm and an oil absorption capacity selected in the range of 1.2-2.5 cc/g.

2. The topical composition according to claim 1, wherein the amount glycerin is selected in the range of 1 to 20 wt.-%, more preferably in the range of 3 to 15 wt.-%, and most preferably in the range of 5 to 10 wt.-%, based on the total weight of the composition.

3. The topical composition according to claim 1, wherein the amount of the silica beads is selected in the range of 0.5 to 10 wt.-%, preferably in the range of 1 to 5 wt.-%, and most preferably in the range of 2 to 4 wt.-%, based on the total weight of the composition.

4. The topical composition according to claim 1, wherein the total amount of microcrystalline cellulose and/or microcrystalline wax is selected in the range of 0.1 to 10 wt.-%, more preferably in the range of 0.5 to 5 wt.-%, based on the total weight of the composition.

5. The topical composition according to claim 1, wherein no microcrystalline wax is present in the topical composition.

6. The topical composition according to claim 1, wherein the oil absorption capacity is selected in the range of in the range of 1.2 to 2.0 cc/g and preferably in the range of 1.3 to 1.8 cc/g.

7. The topical composition according to claim 1, wherein the topical composition further comprises at least one oil soluble UV-filter substance.

8. The topical compositions according to claim 7, wherein the at least one oil soluble UV-filter substance is selected from the group consisting of butyl methoxy-dibenzoylmethane, octocrylene, polysilicone-15 as well as mixtures thereof.

9. The topical composition according to claim 7, wherein the total amount of the at least one additional oil soluble UV-filter substance is selected in the range of 0.1 to 25 wt.-%, preferably in the range of 1 to 20 wt.-%, most preferably in the range of 5 to 15 wt.-%, based on the total weight of the topical composition

10. The topical composition according to claim 7, wherein the composition further comprises at least one inorganic or water soluble UV-filter substance as well as mixtures thereof.

11. The topical composition according to claim 10, wherein the water soluble UV-filter substance is 2-phenylbenzimidazol-sulphonic acid and the inorganic UV-filter substance is titanium dioxide having an inner silica and an outer dimethicone coating.

12. The topical composition according to claim 1, wherein the topical composition is in the form of an oil-in-water (O/W) emulsion comprising an oily phase dispersed in an aqueous phase in the presence of an O/W emulsifier.

13. The topical composition according to claim 12, wherein the O/W emulsifier is potassium cetyl phosphate.

14. Use of silica beads having a particle size Dᵥ0 of greater than 0.3 μm, a Dᵥ100 of less than 35 μm, and a Dᵥ50 selected in the range of 9 to 15 μm and an oil absorption capacity selected in the range of 1.2-2.5 cc/g in combination with microcrystalline cellulose and/or microcrystalline wax to reduce the stickiness of a topical composition comprising glycerin.

15. A method to reduce the stickiness of a topical composition comprising glycerin, said method comprising the step of adding to the topical composition silica beads having a particle size Dᵥ0 of greater than 0.3 μm, a Dᵥ100 of less than 35 μm, a Dv50 selected in the range of 9 to 15 μm and an oil absorption capacity selected in the range of 1.2-2.5 cc/g in combination with microcrystalline cellulose and/or microcrystalline wax and appreciating the effect.
